(19) United States
(12) Patent Application Publication
(10) Pub. No.: US 2021/0340520 A1
(43) Pub. Date: Nov. 4, 2021

(54) METHODS FOR STIMULATING THE PROLIFERATION AND DIFFERENTIATION OF EUKARYOTIC CELLS

(71) Applicants: NANOVIS, LLC, Columbia City, IN (US); PURDUE RESEARCH FOUNDATION, West Lafayette, IN (US)

(72) Inventors: Allen L. GARNER, West Lafayette, IN (US); Ram Anand VADLAMANI, West Lafayette, IN (US); David Alan DETWILER, Columbia City, IN (US); Aginiprakash DHANABAL, West Lafayette, IN (US)

(21) Appl. No.: 17/369,271

(22) Filed: Jul. 7, 2021

Related U.S. Application Data
(63) Continuation of application No. PCT/US2020/013030, filed on Jan. 10, 2020.
(60) Provisional application No. 62/790,865, filed on Jan. 10, 2019.

(51) Int. Cl. C12N 13/00; A61N 1/32; A61N 1/36; A61K 35/32; A61K 35/34; C12N 5/077; A61K 45/06

(52) U.S. Cl. CPC C12N 13/00; A61N 1/326; A61N 1/36171; A61N 1/3616; C12N 2529/00; A61K 35/34; C12N 5/0654; C12N 5/0658; A61K 45/06; A61K 35/32

(57) ABSTRACT

The present disclosure relates to methods of stimulating cell proliferation, promoting differentiation of cells, regenerating cells, promoting nodule formation, and promoting myotube formation. The methods include applying one or more pulses of electricity to cells, each pulse of electricity having a duration of between about 10 nanoseconds and about 1,000 nanoseconds, wherein said pulses of electricity are applied under conditions effective to stimulate cell proliferation, promote differentiation of cells, regenerate cells, promote nodule formation, and promote myotube formation.

This application is a continuation of International Application No. PCT/US2020/013030, filed on Jan. 10, 2020 and published on Jul. 16, 2020, as WO2020/146702, and claims benefit of U.S. Provisional Patent Application Ser. No. 62/790,865, filed Jan. 10, 2019, all of which are hereby incorporated by reference in their entirety.

This invention was made with government support under grant number NRC-HQ-84-14-G-0048 awarded by the U.S. Nuclear Regulatory Commission. The Government has certain rights in the invention.

### FIELD

The present disclosure relates generally to methods for stimulating the proliferation and differentiation of eukaryotic cells by use of electrical pulses.

### BACKGROUND

Low intensity electric fields can induce changes in cell differentiation and cytoskeletal stresses that facilitate manipulation of osteoblasts and mesenchymal stem cells; however, the application times (tens of minutes) are on the order of physiological mechanisms, which can complicate treatment consistency.

While stem cell therapies hold great promise, several challenges remain for clinical translation, including appropriate maintenance of stem cell state, reproducibly expanding large numbers of stem cells for transplantation, efficient differentiation into desired cell types, and ensuring cell viability during and after delivery. For example, the slow proliferation of myoblasts and osteoblasts until differentiation significantly hinders clinical applications for muscular and bone regeneration. Inducing differentiation may benefit certain applications, such as bone healing and regeneration. This has motivated multiple physical methods, such as mechanical and electrical stimulation, and chemical methods, such as substrate and materials design, to control and direct stem cell differentiation and proliferation. Electric fields are increasingly used as an alternative to drugs or gene therapy for treatment and regeneration due to their ease of use and ability to induce desirable phenomena. Electric fields can control differentiation by modifying the membrane potential, which can control voltage gated channels and the influx of ions to determine the differentiation of embryonic stem cells. Electric fields can also induce cytoskeletal stresses to manipulate osteoblasts and mesenchymal stem cells, which was previously possible only by using chemicals or proteins.

Many studies exploring electric field and electromagnetic stimulation of stem cells consider long duration, low intensity electric or magnetic fields. These long duration mechanisms may be challenging to apply consistently because the physical interactions may conflict with long-term physiological mechanisms at similar voltages and currents. For instance, applying a single 2.5 V/cm electric pulse (“EP”) of 90 second duration altered cardiomyocyte differentiation by increasing the number of beating foci while applying a single 5.0 V/cm EP additionally increased intracellular reactive oxygen species. Sauer et al., “Effects of Electrical Fields on Cardiomyocyte Differentiation of Embryonic Stem Cells,” J. Cell Biochem. 75(4):710-23 (1999). A more recent study examined the application of picosecond EPs to manipulate the proliferation and lineage specific gene expression in neural stem cells. Petrella et al., “3D Bioprinter Applied Picosecond Pulsed Electric Fields for Targeted Manipulation of Proliferation and Lineage Specific Gene Expression in Neural Stem Cells,” J. Neural Eng. 15(5):056021 (2018).

Although the effect of these electric fields on osseointegration are incompletely characterized, recent studies have shown that electrical stimulation can enhance bone growth. Applying voltages under 500 mV to the titanium surfaces utilized in implants clinically promoted bone regeneration for fractures by enhancing osteoblast differentiation. Gittens et al., “Electrical Polarization of Titanium Surfaces For the Enhancement of Osteoblast Differentiation,” Bioelectromagnetics 34(8):599-612 (2013). Applying degenerate sine-wave and capacitively coupled stimulation for 4 hours increased differentiation and mineralization and collagen production of osteoblast-like cells in vitro. Griffen et al., “Enhancement of Differentiation and Mineralisation of Osteoblast-like Cells by Degenerate Electrical Waveform in an In Vitro Electrical Stimulation Model Compared to Capacitive Coupling,” PLOS ONE 8(9):e72978 (2013). Electrical stimulation increased the growth of adipose-derived mesenchymal stem cells in conductive scaffolds by manipulating voltage-gated calcium, sodium and potassium channels. Zhang et al., “Electrical Stimulation of Adipose-Derived Mesenchymal Stem Cells in Conductive Scaffolds and the Roles of Voltage-Gated Ion Channels,” Acta Biomater. 32:46-56 (2016).

Adult skeletal muscle demonstrates an efficient regenerative capacity in response to physiological stimulus, such as intense exercise and muscle injury, by activating resident stem cells (satellite cells) in a mediated myogenic program. These cells remain quiescent between the basal lamina and the plasma membrane of the myofibers until activated by regenerative signals. Once stimulated, these satellite cells undergo multiple rounds of divisions, differentiation, and fusion to form new multinucleated myofibers, which is critical for postnatal maintenance of skeletal muscle and muscle repair. Aging muscles exhibit impaired regenerative ability, partly due to a loss of stem cell populations and increased defects in satellite cells.

While nanosecond electric pulses have been utilized for treatment before, such as inactivating microorganisms and activating apoptotic pathways in melanomas, the application of nanosecond electric pulses with a lower cumulative energy density on stem cell stimulation is lacking. Electric field intensity can dramatically impact mechanism. Recent studies using electrostimulation with capacitive coupling (indirect contact with a sample) induced similar levels of hematopoietic and mesenchymal stem cell activation as bovine thrombin, the state of the art platelet activator, while conductive coupling (direct contact with the sample) increased cell death. Although the applied voltage was the same, capacitive coupling induces a much lower membrane potential than conductive coupling, creating less intense biophysical effects. In general, applying greater pulse energy (more pulses, higher electric field, or longer pulse duration) induces cell death.

There remains a need to improve methods of stimulating proliferation and differentiation of cells. The present disclosure is directed to overcoming these and other deficiencies in the art.

### SUMMARY

A first aspect relates to a method of stimulating cell proliferation. The method includes applying one or more pulses of electricity to cells, each pulse of electricity having a duration of between about 10 nanoseconds and about 1,000 nanoseconds, wherein said pulses of electricity are applied under conditions effective to stimulate cell proliferation.

A second aspect relates to a method of promoting differentiation of cells. The method includes applying one or more pulses of electricity to cells, each pulse of electricity having a duration of between about 10 nanoseconds and about 1,000 nanoseconds, wherein said pulses of electricity are applied under conditions effective to promote differentiation of cells.

A third aspect relates to a method of regenerating cells. The method includes applying one or more pulses of electricity to cells, each pulse of electricity having a duration of between about 10 nanoseconds and about 1,000 nanoseconds, wherein said pulses of electricity are applied under conditions effective to promote cell regeneration.

A fourth aspect relates to a method of promoting nodule formation. The method includes applying one or more pulses of electricity to cells, each pulse of electricity having a duration of between about 10 nanoseconds and about 1,000 nanoseconds, wherein said pulses of electricity are applied under conditions effective to promote nodule formation.

A fifth aspect relates to a method of promoting myotube formation. The method includes applying one or more pulses of electricity to cells, each pulse of electricity having a duration of between about 10 nanoseconds and about 1,000 nanoseconds, wherein said pulses of electricity are applied under conditions effective to promote myotube formation.

Low intensity electrical fields can induce changes in cell differentiation and cytoskeletal stresses that facilitate manipulation of osteoblasts and mesenchymal stem cells; however, the application times (tens of minutes) are on the order of physiological mechanisms, which can complicate treatment consistency. Intense nanosecond electrical pulses (“NSEPs”) can overcome these side effects by inducing similar stresses on shorter timescales while additionally inducing plasma membrane nanoporation, ion transport, and intracellular structure manipulation.

The present disclosure shows that treating myoblasts and osteoblasts with five 300 nanosecond electric pulses (“NSEPs” or “nanosecond EPs”) with intensities from 1.5 to 25 kV/cm increased proliferation and differentiation. While myoblast population decreased for NSEPs above 5 kV/cm, it increased by approximately five-fold 48 hours after exposure to 10 kV/cm and 20 kV/cm trains when all cell concentrations were fixed to the same level after exposure. Three trials of NSEP-treated osteoblasts showed that NSEP trains between 2.5 kV/cm and 10 kV/cm induced the greatest population growth compared to the control 48 hours after treatment. NSEP trains between 1.5 kV/cm and 5 kV/cm induced the most nodule formation in osteoblasts, indicating bone formation. These results demonstrate the potential utility for NSEPs to rapidly modulate stem cells for proliferation and differentiation and motivate further experiments on parameter optimization for in vivo applications.

### BRIEF DESCRIPTION OF THE DRAWINGS

FIG. 1 shows an electric field for a representative 300 nanosecond (“ns”) pulse produced by the pulse generator across a 0.2 cm cuvette by using E=VID with V the applied voltage and D the cuvette gap, in accordance with an aspect of the present invention;

FIGS. 2A-2B show cell count immediately after treatment to plates with an identical number of cells in each well (FIG. 2A) and myoblast population determined by MTT assays 24 and 48 hours after five 300 ns electric pulses (FIG. 2B), in accordance with an aspect of the present invention;

FIG. 3 shows representative immunostaining images from three different wells of untreated myoblasts (top) and myoblasts following exposure to five 5 kV/cm (middle) or 25 kV/cm, 300 ns (bottom) electric pulses. The red and blue mark the myosin heavy chains and cell nuclei, respectively. The myoblasts treated with 5 kV/cm have a larger concentration of red cells, indicating increased proliferation and differentiation. The 25 kV/cm treatment induced lowered cell counts and differentiation, in accordance with an aspect of the present invention;

FIGS. 4A-4C show osteoblast proliferation: FIG. 4A depicts osteoblast proliferation 0, 24, and 48 hours as a percentage of the initial untreated control population after treatment with 300 ns EPs with various electric fields for three different trials demonstrating increased proliferation compared to untreated control (0 kV/cm) for electric fields from (a) 10 to 20 kV/cm at 24 hours and 2.5 to 20 kV/cm at 48 hours after treatment; FIG. 4B shows osteoblast proliferation 0, 24 and 48 hours as a percentage of the initial untreated control population after treatment with 300 ns EPs with various electric fields for three different trials demonstrating increased proliferation compared to untreated control (0 kV/cm) for electric fields from 2.5 to 20 kV/cm at 48 hours, (b) 2.5 to 20 kV/cm at 24 hours and 2.5 to 20 kV/cm at 48 hours after treatment; and FIG. 4C shows osteoblast proliferation 0, 24, and 48 hours as a percentage of the initial untreated control population after treatment with 300 ns EPs with various electric fields for three different trials demonstrating increased proliferation compared to untreated control (0 kV/cm) for electric fields from 2.5 to 10 kV/cm at 48 hours after treatment, in accordance with an aspect of the present invention;

FIG. 5 depicts representative fluorescent images from each of three wells for untreated osteoblast control cells 7 days (top) and 14 days (bottom) after the experiment indicating light nodule formation after 14 days, as indicated by the red coloration, in accordance with an aspect of the present invention;

FIG. 6 shows representative fluorescent images from each of three wells 7 days (top) and 14 days (bottom) after treating osteoblast cells with five, 1.5 kV/cm, 300 ns electric pulses 7 days and 14 days indicating enhanced nodule formation after 14 days, in accordance with an aspect of the present invention;

FIG. 7 depicts representative fluorescent images from each of three wells 7 days (top) and 14 days (bottom) after treating osteoblast cells with five, 2.5 kV/cm, 300 ns electric pulses indicating nodule formation after 7 days and more extensive nodule formation after 14 days, in accordance with an aspect of the present invention;

FIG. 8 shows representative fluorescent images from each of three wells 7 days (top) and 14 days (bottom) after treating osteoblast cells with five, 5 kV/cm, 300 ns electric pulses indicating nodule formation after 7 days and more extensive nodule formation after 14 days, in accordance with an aspect of the present invention; and

FIG. 9 shows representative fluorescent images from each of three wells 7 days (top) and 14 days (bottom) after treating osteoblast cells with five, 10 kV/cm, 300 ns electric pulses showing nodule formation after 7 days and more extensive nodule formation after 14 days, in accordance with an aspect of the present invention.

### DETAILED DESCRIPTION

A first aspect relates to a method of stimulating cell proliferation. The method includes applying one or more pulses of electricity to cells, each pulse of electricity having a duration of between about 10 nanoseconds and about 1,000 nanoseconds, wherein said pulses of electricity are applied under conditions effective to stimulate cell proliferation.

A second aspect relates to a method of promoting differentiation of cells. The method includes applying one or more pulses of electricity to cells, each pulse of electricity having a duration of between about 10 nanoseconds and about 1,000 nanoseconds, wherein said pulses of electricity are applied under conditions effective to promote differentiation of cells.

It is to be appreciated that certain aspects, modes, embodiments, variations and features of the present invention are described below in various levels of detail in order to provide a substantial understanding of the present technology. The definitions of certain terms as used in this specification are provided below. Unless defined otherwise, all technical and scientific terms used herein generally have the same meaning as commonly understood by one of ordinary skill in the art to which this invention belongs.

As used herein, the term “about” means that the numerical value is approximate and small variations would not significantly affect the practice of the disclosed embodiments. Where a numerical limitation is used, unless indicated otherwise by the context, “about” means the numerical value can vary by ±10% and remain within the scope of the disclosed embodiments.

As used herein, the terms “subject,” “individual” or “patient,” used interchangeably, means any animal, including mammals, such as mice, rats, other rodents, rabbits, dogs, cats, swine, cattle, sheep, horses, or primates, such as humans.

As used herein, the term “purified” means that when isolated, the isolate contains at least 90%, at least 95%, at least 98%, or at least 99% of a compound described herein by weight of the isolate.

As used herein, the phrase “substantially isolated” means a compound that is at least partially or substantially separated from the environment in which it is formed or detected.

It is further appreciated that certain features described herein, which are, for clarity, described in the context of separate embodiments, can also be provided in combination in a single embodiment. Conversely, various features which are, for brevity, described in the context of a single embodiment, can also be provided separately or in any suitable sub-combination.

A “nanosecond electric pulse” or a “sub-microsecond electric pulse”, sometimes abbreviated as NSEP or nsEP, refers to an electrical pulse with a width of between 0.1 nanoseconds (“ns”) to 1000 nanoseconds, or as otherwise known in the art. A plurality of nanosecond electric pulses may be used to generate a nanosecond pulsed electric field. Electric pulses having nanosecond duration (such as electric pulses with a duration of between about 1 nanosecond and about 1 microsecond (e.g., 1,000 nanoseconds)) are referred to herein interchangeably as “nanosecond electric pulse(s)” and “nanosecond-duration EP(s)” (abbreviated herein as “NSEP”, “ns EP”, “ns electric pulses”, and “nanosecond EP”). Nanosecond electric pulses (NSEPs) refer to an electrical pulse with a width of between 0.1 nanoseconds (ns) to 1000 nanoseconds, or as otherwise known in the art.

Technology development over the past two decades has led to the biomedical application of nanosecond-duration EPs (NSEPs) (such as EPs with a duration of between about 1 nanosecond and about 1 microsecond) with field strengths ranging from tens of kV/cm to a few hundred kV/cm. These shorter durations enable charging intracellular membranes prior to the cell membrane, permitting intracellular manipulation with minimal cell membrane impact. Without being limited to any particular theory of activity, NSEPs may also permit creating membrane nanopores that enable ions and small molecules to enter the cell while prohibiting larger molecules.

An abbreviated time during which NSEPs may be applied in order to increase cell proliferation and cell differentiation may advantageously facilitate treatment. For example, NSEPs, in one embodiment, may be applied to a subject (e.g., human or animal) over a period of an hour or less (or 30 minutes or less, or 20 minutes or less, or 15 minutes or less, or 10 minutes or less, or 5 minutes or less, or two minutes or less, or one minute or less), or any range of time therebetween. In one embodiment, NSEPs may be administered for one hour or less. For example NSEPs may be administered for a period of time in a range between less than about 1 nanosecond, more than about 1 nanosecond, less than about 1 second, less than about 5 seconds, less than about 10 seconds, about 10 seconds to about 30 seconds, about 10 seconds to one about 60 seconds, between about 1 second and 60 seconds, between about one minute and 60 minutes, between about 1 minute and about 45 minutes, between about 1 minute and about 30 minutes, between about 1 minute and about 15 minutes, and between about one minute and about 10 minutes, or any period of time or range of time therebetween. In another embodiment, NSEPs may be administered across a duration of time in excess of an hour. For example, NSEPs may be administered for two, three four, five, six, seven, eight, nine, ten, eleven, twelve, or more hours.

The NSEPs described herein may be administered according to various parameters. Such parameters include, for example, intensity of NSEP applied, duration of NSEP, frequency of NSEP administration, number of NSEPs applied in a train of NSEPs, number of trains of NSEPs applied, and duration of time between trains of NSEPs. In one embodiment, the intensity of NSEPs may be within a range of about 1 kV/cm to about 50 kV/cm. As described herein, “about” describes that the intensity of NSEPs may vary somewhat from these precise values while still falling within the intensities as so described. For example, “about” may mean within +/−5% of a value or within +/−10% of a value. Intensity may be within such range, or within a sub-range thereof. For example, intensity of an NSEP may be about 1 kV/cm, about 2 kV/cm, about 3 kV/cm, about 4 kV/cm, about 5 kV/cm, about 6 kV/cm, about 7 kV/cm, about 8 kV/cm, about 9 kV/cm, about 10 kV/cm, about 15 kV/cm, about 20 kV/cm, about 25 kV/cm, about 30 kV/cm, about 35 kV/cm, about 40 kV/cm, about 45 kV/cm, and about 50 kV/cm. In one embodiment, each pulse of electricity has an intensity peak of about 1.0 kV/cm. In another embodiment, each pulse of electricity has an intensity peak in a range of about 1.0 kV/cm to about 30.0 kV/cm. NSEPs may be, for example, within a range from about 1.0 kV/cm to about 30.0 kV/cm, from about 5 kV/cm to about 30 kV/cm, from about 10 kV/cm to about 25 kV/cm, from about 15 kV/cm to about 20 kV/cm, from about 10 kV/cm to about 35 kV/cm, from about 10 kV/cm to about 30 kV/cm, from about 10 kV/cm to about 25 kV/cm, from about 10 kV/cm to about 20 kV/cm, from about 10 kV/cm to about 15 kV/cm, from about 1 kV/cm to about 25 kV/cm, from about 5.0 kV/cm to about 10 kV/cm, or within any subranges within these ranges. In one embodiment, each pulse of electricity has an intensity peak in a range of between about 1.0 kV/cm and about 25.0 kV/cm. In one embodiment, each pulse of electricity has an intensity peak in a range of about 5.0 kV/cm and about 10.0 kV/cm. In one embodiment, each pulse of electricity has an intensity peak in a range of about 2.5 kV/cm to about 25.0 kV/cm. Alternatively, the NSEPs may have an intensity below about 20 kV/cm. For example, an EP may have an intensity of about 15 kV/cm, or about 10 kV/cm, or about 5 kV/cm, or about 1 kV/cm, or any value therebetween. In other examples, an NSEP may have an intensity above about 30 kV/cm. For example, an NSEP may have an intensity of about 35 kV/cm, or about 40 kV/cm, or about 45 kV/cm, or about 50 kV/cm, or about 60 kV/cm, or about 70 kV/cm, or about 75 kV/cm, or about 80 kV/cm, or about 85 kV/cm, or about 90 kV/cm, or about 100 kV/cm, or any value or range therebetween. In one embodiment, NSEP has an intensity between about 1.5 to about 25 kV/cm, or between about 10 kV/cm and about 20 kV/cm, or between about 2.5 kV/cm and about 10 kV/cm, or between about 1.5 kV/cm and about 5 kV/cm.

The NSEPs may have a duration of between about 1 nanosecond (“ns”) and about 1,000 ns (i.e., 1 microsecond). In this case, “about” means duration of EPs may vary somewhat from these precise values while still falling within the intensities as so described. For example, “about” may mean within +/−5% of a value or within +/−10% of a value. In another embodiment, the NSEPs may be between about 50 ns and about 300 ns, or any value or range therebetween. An NSEP may, in one embodiment, have a duration of about 1 ns, or about 10 ns, or about 20 ns, or about 30 ns, or about 40 ns, or about 50 ns, or about 60 ns, or 70 ns, or about 80 ns, or about 90 ns, or about 100 ns, or about 110 ns, or about 120 ns, or about 130 ns, or about 140 ns, or about 150 ns, or about 160 ns, or about 170 ns, or about 180 ns, or about 190 ns, or about 200 ns, or about 210 ns, or about 220 ns, or about 230 ns, or about 240 ns, or about 250 ns, or about 260 ns, or about 270 ns, or about 280 ns, or about 290 ns, or about 300 ns, or about 310 ns, or about 320 ns, or about 330 ns, or about 340 ns, or about 350 ns, or about 360 ns, or about 370 ns, or about 380 ns, or about 390 ns, or about 400 ns, or about 410 ns, or about 420 ns, or about 430 ns, or about 440 ns, or about 450 ns, or about 460 ns, or about 470 ns, or about 480 ns, or about 490 ns, or about 500 ns, or about 510 ns, or about 520 ns, or about 530 ns, or about 540 ns, or about 550 ns, or about 560 ns, or about 570 ns, or about 580 ns, or about 590 ns, or about 600 ns, or about 610 ns, or about 620 ns, or about 630 ns, or about 640 ns, or about 650 ns, or about 660 ns, or about 670 ns, or about 680 ns, or about 690 ns, or about 700 ns, or about 710 ns, or about 720 ns, or about 730 ns, or about 740 ns, or about 750 ns, or about 760 ns, or about 770 ns, or about 780 ns, or about 790 ns, or about 800 ns, or about 810 ns, or about 820 ns, or about 830 ns, or about 840 ns, or about 850 ns, or about 860 ns, or about 870 ns, or about 880 ns, or about 890 ns, or about 900 ns, or about 910 ns, or about 920 ns, or about 930 ns, or about 940 ns, or about 950 ns, or about 960 ns, or about 970 ns, or about 980 ns, or about 1,000 ns. In one embodiment, the NSEPs may be a duration of between about 10 nanoseconds and about 300 nanoseconds, or any value or range therebetween.

An NSEP may also have a duration within any subrange within about 50 ns to about 300 ns. For example, an NSEP may have a duration of between about 50 ns and 100 ns, about 50 ns and about 250 ns, about 50 ns and about 200 ns, about 50 ns and about 150 ns, about 50 ns and about 100 ns, about 100 ns and about 150 ns, about 150 ns and about 200 ns, about 250 ns and about 300 ns, about 100 ns and about 200 ns, about 200 ns and about 300 ns, about 100 ns and about 300 ns, and any value or range therebetween.

In some embodiments, NSEPs may have a duration of less than about 50 ns. For example, an NSEP may have a duration of about 45 ns, about 40 ns, about 35 ns, about 30 ns, about 25 ns, about 20 ns, about 15 ns, about 10 ns, about 5 ns, or about 1 ns, or a duration within a range therebetween. NSEPs have a duration of less than 1 microsecond (μs).

NSEPs may be administered in a series or train of NSEPs, meaning more than one NSEP may be applied in temporally proximate succession. For example, anywhere from 2 to 200 NSEPs may be applied in a train with a frequency of administration of between about 0.01 Hz and about 1,000 Hz. In this case, “about” means frequency of NSEPs may vary somewhat from these precise values while still falling within the intensities as so described. For example, 2, about 3, about 4, about 5, about 6, about 7, about 8, about 9, about 10, about 12, about 15, about 17, about 20, about 22, about 25, about 27, about 30, about 32, about 35, about 37, about 40, about 42, about 45, about 47, about 50, about 52, about 55, about 57, about 60, about 62, about 65, about 67, about 70, about 72, about 75, about 77, about 80, about 82, about 85, about 87, about 90, about 92, about 95, about 97, about 100, about 102, about 105, about 107, about 110, about 112, about 15, about 117, about 120, about 122, about 125, about 127, about 130, about 132, about 135, about 137, about 140, about 142, about 145, about 147, about 150, about 152, about 155, about 157, about 160, about 162, about 165, about 167, about 170, about 172, about 175, about 177, about 180, about 182, about 185, about 187, about 190, about 192, about 195, about 197, or about 200 NSEPs may be administered in a train of between 0.01 Hz and about 1,000 Hz. In this embodiment, “about” means the number of EPs may be within up to +/−10 of the number indicated. Any number of NSEPs or subrange within the foregoing identified number of NSEPs may also be applied. In one embodiment, between about 15 and about 20, about 10 and about 40, or about 20 and about 100 NSEPs may be administered at a frequency of between about 0.01 Hz and about 1,000 Hz.

In another example, anywhere from 2 to 1,000 NSEP may be applied in temporally proximate succession. For example, anywhere from 2 to 1,000 NSEPs may be applied in a train with a frequency of administration of between about 0.01 Hz and about 1,000 Hz. In this embodiment, “about” means frequency of NSEPs may vary somewhat from these precise values while still falling within the intensities as so described. For example, about 1, about 2, about 3, about 4, about 5, about 6, about 7, about 8, about 9, about 10, about 50, about 75, about 100, about 125, about 150, about 175, about 200, about 250, about 300, about 350, about 400, about 450, about 500, about 550, about 600, about 650, about 700, about 750, about 800, about 850, about 900, about 950, about 1,000, about 1,050, about 1,100, about 1,150, about 1,200, about 1,250, about 1,300, about 1,350, about 1,400, about 1,450, about 1,500, about 1,550, about 1,600, about 1,650, about 1,700, about 1,750, about 1,800, about 1,850, about 1,900, about 1,950, about 2,000, about 2,050, about 2,150, about 2,100, about 2,150, about 2,200, about 2,250, about 2,300, about 2,350, about 2,400, about 2,450, about 2,500, about 2,550, about 2,600, about 2,650, about 2,700, about 2,750, about 2,800, about 2,850, about 2,900, about 2,950, about 2,300, about 2,350, about 2,400, about 2,450, about 2,500, about 2,550, about 2,600, about 2,650, about 2,700, about 2,750, about 2,800, about 2,850, about 2,900, about 2,950, or about 3,000 EPs may be administered in a train of between 0.01 Hz and about 1,000 Hz. In this embodiment, “about” means the number of NSEPs may be within +/−25 of the number indicated. In another example, more than about 3,000 NSEPs may be administered (such as about 4,000 or about 5,000 or more). Any number of NSEPs or subrange within the foregoing identified number of NSEPs may also be applied. In an example, between about 100 and about 500, about 400 and about 800, or about 600 and about 1,000 NSEPs may be administered at a frequency of between about 0.01 Hz and about 1,000 Hz. In one embodiment, up to 150 pulses of electricity are applied, for example, about 10 pulses, about 20 pulses, about 30 pulses, about 40 pulses, about 50 pulses, about 60 pulses, about 70 pulses, about 80 pulses, about 90 pulses, about 100 pulses, about 110 pulses, about 120 pulses, about 130 pulses, about 140 pulses, about 150 pulses, or any value or range therebetween. In one embodiment, five or fewer pulses of electricity are applied.

In one embodiment, the frequency of administration of NSEPs may be as low as about 0.01 Hz and as high as about 1,000 Hz. In one embodiment, frequency is about 1 Hz. In another embodiment, frequency is between about 0.01 Hz and about 1,000 Hz. In another embodiment, frequency may be about 0.01 Hz, about 0.02 Hz, about 0.03 Hz, about 0.4 Hz, about 0.05 Hz, about 0.06 Hz, about 0.07 Hz, about 0.08 Hz, about 0.09 Hz, about 0.1 Hz, about 0.5 Hz, about 1.0 Hz, about 1.5 Hz, about 2.0 Hz, about 2.5 Hz, about 3.0 Hz, about 3.5 Hz, about 4.0 Hz, about 4.5 Hz, about 5.0 Hz, about 5.5 Hz, about 6.0 Hz, about 6.5 Hz, about 7.0 Hz, about 7.5 Hz, about 8.0 Hz, about 8.5 Hz, about 9.0 Hz, about 9.5 Hz, about 10 Hz, about 25 Hz, about 30 Hz, about 35 Hz, about 40 Hz, about 45 Hz, about 50 Hz, about 55 Hz, about 60 Hz, about 65 Hz, about 70 Hz, about 75 Hz, about 80 Hz, about 85 Hz, about 90 Hz, about 95 Hz, about 100 Hz, about 110 Hz, about 125 Hz, about 150 Hz, about 175 Hz, about 200 Hz, about 225 Hz, about 250 Hz, about 275 Hz, about 300 Hz, and any value or range therebetween. Trains of pulses may also be administered within a range of pulses overlapping these frequencies. For example, in one embodiment, each pulse of electricity has a frequency of repetition in a range of between about 0.1 Hz to about 300 Hz. In another embodiment, wherein each pulse of electricity has a frequency of repetition in a range of between about 0.5 Hz to about 10 Hz. In yet another embodiment, a train of NSEPs may be administered at a frequency of between about 1 Hz and about 2 Hz, about 1 Hz and about 3 Hz, about 1 Hz and about 5 Hz, about 3 Hz and about 5 Hz, about 5 Hz and about 7.5 Hz, about 5 Hz and about 10 Hz, about 1 Hz and about 50 Hz, about 1 Hz and about 75 Hz, about 1 Hz and about 100 Hz, about 1 Hz and about 150 Hz, about 1 Hz and about 200 Hz, about 1 Hz and about 250 Hz, and about 1 Hz and about 300 Hz.

In some embodiments, NSEPs may have a duration of between about 10 ns and about 300 ns, may have an intensity of between about 1.0 kV/cm and about 50 kV/cm or between about 1.0 kV/cm and about 30 kV/cm, and be administered at between about 0.01 Hz and about 1,000 Hz in a train of between 10 to 20 NSEPs administered. However, explicitly included within the present disclosure is different combinations of the foregoing parameters. Administration of any number of NSEPs within a train as disclosed herein having a duration of between about 10 ns and about 1,000 ns, an intensity of between about 1.0 kV/cm and about 50 kV/cm, and administered at between about 0.01 Hz and about 300 Hz, is for example, within the embodiments described herein.

NSEPs may be generated by a Blumlein circuit, which can be built in numerous configurations using capacitors (based on capacitance/charge storage devices), including, but not limited to, ceramic based capacitors, transmission lines, and other dielectrics (such as water). One can control the NSEP duration by the Blumlein circuit design either by controlling the capacitance or length of the transmission line. Similarly, one can control the pulse shape by modifying the number and nature of the switches to vary the rise- and fall-times, which influences whether the pulse appears square or trapezoidal with respect to time. Increasing the voltage beyond the physical capabilities of the materials used in the Blumlein circuit can be achieved by using a Marx generator, which is a voltage adding device. Typical Marx approaches charge parallel full-bridge switch-capacitor cells at a lower voltage, and through controllable switches, connect in series with a biological sample and discharge into the load at a higher voltage as a function of the number of series components. The resulting series equivalent capacitor (“Ceq”) voltage is discharged into the biological load, which is calculated as Vload NVc where N is the number of Marx stages with capacitors charged to Vc and depends on stray system capacitance and inductance. Various pulse generator designs that may be relevant, including a modular, controllable Marx-based technology developed in collaboration with GE particularly for platelet activation are described in Garner et al., “Design, Characterization and Experimental Validation of a Compact, Flexible Pulsed Power Architecture for Ex Vivo Platelet Activation,” PLOS ONE 12(7):e0181214 (2017) and U.S. Pat. No. 9,238,808 to Caiafa et al., both of which are hereby incorporated by reference in their entirety.

In one embodiment, more than one train of NSEPs may be administered. For example, two, three, four, five, six, or more trains may be administered. A duration between trains may be anywhere from between one minute to about one hour. In some embodiments, the duration between trains may be 5 min, 10 min, 15 min, 20 min, 25 min, 30 min, 35 min, 40 min, 45 min, 50 min, 55 min, or 60 min. In one embodiment, administering more than one train of NSEPs within a duration between trains of 60 min or less, such as 30 min or less or 20 min or less of 15 min or less or 10 min or less or 5 min or less, may advantageously enhance an effect of a method as disclosed herein by application of more than one train of NSEPs but within an abbreviated time frame for greater ease and improved logistics of application. Thus, treatment times on the order of minutes in accordance with the present disclosure may replace what conventional therapy may require hours or days to attain. In other examples, longer durations between trains may be used when desirable or advantageous or where shorted inter-train intervals are not required or desired. In one embodiment, trains may be separated by about 15 min or 20 min. And of the durations of inter-train intervals as disclosed herein may be about or approximately of the durations identified, in that they may be +/−5% or +/−10% of the duration indicated.

In one embodiment, a 30 nanosecond rise and fall time is present between a peak intensity and a baseline intensity. A peak intensity as described herein refers to the highest intensity of a NSEP. A baseline intensity as described herein refers to the intensity value when zero NSEP is applied. The rise and fall time between the peak intensity and baseline intensity may, alternatively, be under 30 nanoseconds or may be above 30 nanoseconds. For example, the rise and fall time between the peak intensity and baseline intensity may be about 5 nanoseconds, about 10 nanoseconds, about 15 nanoseconds, about 20 nanoseconds, about 25 nanoseconds, about 30 nanoseconds, about 35 nanoseconds, about 40 nanoseconds, about 45 nanoseconds, about 50 nanoseconds, about 55 nanoseconds, about 60 nanoseconds, about 65 nanoseconds, about 70 nanoseconds, about 75 nanoseconds, about 80 nanoseconds, about 85 nanoseconds, about 90 nanoseconds, about 95 nanoseconds, about 100 nanoseconds, about 125 nanoseconds, about 150 nanoseconds, about 175 nanoseconds, about 200 nanoseconds, about 250 nanoseconds, about 300 nanoseconds, and may be above 300 nanoseconds, or any value or range therebetween.

In one embodiment, there is a time between rise and fall times of a peak intensity and a baseline intensity of less than about 10 nanoseconds. Alternatively, the time between rise and fall times of a peak intensity and a baseline intensity may be more than about 10 nanoseconds. For example, the time between rise and fall times of a peak intensity and a baseline intensity may be less than about 1 nanosecond, about 1 nanosecond, about 2 nanoseconds, about 3 nanoseconds, about 4 nanoseconds, about 5 nanoseconds, about 6 nanoseconds, about 7 nanoseconds, about 8 nanoseconds, about 9 nanoseconds, about 10 nanoseconds, about 15 nanoseconds, about 20 nanoseconds, about 30 nanoseconds, about 45 nanoseconds, about 60 nanoseconds, greater than about 60 nanoseconds, or any value or range therebetween.

Provided in the present disclosure is a method for improving cell proliferation and cell differentiation. For example, by treating myoblasts and osteoblasts with five 300 ns electric pulses (NSEPs) with intensities from 1.5 to 25 kV/cm, cell proliferation and differentiation may increase. 10 kV/cm and 20 kV/cm trains may, in one embodiment, increase myoblast population by approximately five-fold 48 hours after exposure when all cell densities were set to the same level after exposure. In one embodiment, NSEP-treated osteoblasts after exposure to NSEP trains between 2.5 kV/cm and 10 kV/cm, may induce population growth compared to a control sample over a period of time, for example, 48 hours after treatment. In one embodiment, NSEP trains between 1.5 kV/cm and 5 kV/cm may induce nodule formation in osteoblasts, indicative of bone formation. The present disclosure, thus, demonstrates the potential utility for NSEPs to rapidly modulate stem cells for proliferation and differentiation and in in vitro and in vivo applications.

In one embodiment, the impact of nanosecond electric pulses (NSEPs) on osteoblast and myoblast proliferation and differentiation is assessed. NSEPs may, in some embodiments, avoid potential challenges of low voltage electric fields by applying decisively non-physiological parameters (electric fields of 30-300 kV/cm and pulse durations of 10-300 ns) to induce various physical mechanisms, such as plasma membrane nanoporation, ion transport, and intracellular structure manipulation. NSEPs may, in one embodiment, induce these phenomena with minimal tissue heating and the ability to target intracellular structures, such as calcium stores and the cytoskeleton. In one embodiment, appropriate tuning of intense NSEPs provides the potential to provide both mechanical and electrical stresses to facilitate adequate microenvironment control to manipulate stem cell function.

In one embodiment, the application of NSEPs are applied having a low cumulative energy density on stem cell stimulation. Electric field intensity can dramatically impact mechanism. Prior to the discovery of the present disclosure, it was generally considered in the art that applying greater pulse energy (more pulses, higher electric field, or longer pulse duration) induces cell death. The present disclosure, in one embodiment, may select NSEP parameters to stimulate osteoblast and myoblast behavior without inducing adverse effects, such as cell death, much as EPs are applied for platelet activation. While applying NSEPs allows for a lower duty cycle and application of higher electric fields, this disclosure, in one embodiment, demonstrates that applying only five NSEPs from 2.5 kV/cm to 5 kV/cm stimulates osteoblasts and myoblasts with potential implications to regenerative healing and tissue repair.

Cells that may be utilized in accordance with the present disclosure include any cell in need of or any cell that may benefit from increased proliferation or differentiation. For example, the method may be applied to stem cells, satellite cells, myoblasts, osteoblasts, chondrocytes, fibroblasts, tenocytes, precursor cells, embryological cells, progenitor cells, mesenchymal stem cells, neural stem cells, glial progenitor cells, angioblast hematopoietic stem cells, induced pluripotent stem cells, allograft stem cells, and xenograft stem cells. Adult skeletal muscle, which contains cells utilized in some embodiments of the present disclosure, demonstrates an efficient regenerative capacity in response to physiological stimulus, such as intense exercise and muscle injury, by activating resident stem cells (satellite cells) in a mediated myogenic program. These cells remain quiescent between the basal lamina and the plasma membrane of the myofibers until activated by regenerative signals. Once stimulated, these satellite cells may undergo multiple rounds of divisions, differentiation and fusion to form new multinucleated myofibers, which is critical for postnatal maintenance of skeletal muscle and muscle repair. Aging muscles, which contains cells utilized in some embodiments of the present disclosure, exhibit impaired regenerative ability, partly due to a loss of stem cell populations and increased defects in satellite cells.

In one embodiment, the electric pulses are applied to cells in vivo. In such an embodiment, the methods of the present disclosure may involve selecting a subject based on levels of a particular cell type during a time period where increased cell proliferation or differentiation, or increased muscle/bone generation or regeneration is sought, compared to a reference level for a subject not having a need or desire for increased cell proliferation or differentiation, or increased muscular/bone generation or regeneration. As used herein, the term “reference level” refers to an amount of a substance, e.g., particular cell type (for example, stem cells), which may be of interest for comparative purposes. In some embodiments, a reference level may be the level or concentration of a population of a cell type expressed as an average of the level or concentration from samples of a control population of healthy (disease-free and/or pathogen-free) subjects. In other embodiments, the reference level may be the level in the same subject at a different time, e.g., before the present invention is employed, such as the level determined prior to the subject developing a disease, disease condition, and/or pathogenic infection, prior to initiating therapy, such as, for example, stem cell therapy, or earlier in the therapy. Mammalian subjects according to this aspect of the present invention include, for example, human subjects, equine subjects, porcine subjects, feline subjects, and canine subjects. Human subjects are particularly preferred.

Exemplary methods of comparing cell population levels between a subject and a reference level include, but are not limited to, comparing differences in detected cell population levels, based on results of one or more assays (e.g., a cell proliferation assay). In some embodiments, cell population levels are lower in the presence of a muscular or bone disease or muscular or bone injury than in a subject having no muscular or bone disease or no muscular or bone injury.

As used herein, the phrase “therapeutically effective amount” means an amount of active compound or pharmaceutical agent that elicits the biological or medicinal response that is being sought in a tissue, system, animal, individual or human by a researcher, veterinarian, medical doctor or other clinician. The therapeutic effect is dependent upon the disorder being treated or the biological effect desired. As such, the therapeutic effect can be a decrease in the severity of symptoms associated with the disorder and/or inhibition (partial or complete) of progression of the disorder, or improved treatment, healing, prevention or elimination of a disorder, or side-effects. The amount needed to elicit the therapeutic response can be determined based on the age, health, size and sex of the subject. Optimal amounts can also be determined based on monitoring of the subject's response to treatment.

In one embodiment, the electrical pulses are applied to cells in vitro. In one embodiment, the method further includes inserting the cells into a subject following applying of one or more electrical pulses. In this embodiment, a cell population can be taken from a subject or from a second subject then administered to a first subject (e.g., by injecting the cell population into the first subject).

In all embodiments that involve applying the one or more pulses of electricity to cells from a subject, any combination of administration can be accomplished either via systemic administration to the subject or via targeted administration to affected tissues, organs, and/or cells. The cell population following application of NSEPs may be administered to a non-targeted area along with one or more agents that facilitate migration of the cells (and/or uptake by) a targeted tissue, organ, or cell. Additionally and/or alternatively, the cells themselves can be modified to facilitate transport to (and uptake by) the desired tissue, organ, or cell, as will be apparent to one of ordinary skill in the art.

In one embodiment, an additional agent may be administered to the cells in addition to the one or more pulses of electricity.

As used herein, the term “simultaneous” therapeutic use refers to the administration of at least one additional agent beyond the one or more pulses of electricity (e.g., NSEPs), optionally, by the same route and at the same time or at substantially the same time. As used herein, the term “separate” therapeutic use refers to an administration of at least one additional agent beyond the one or more pulses of electricity (e.g., NSEPs) ingredients at the same time or at substantially the same time by different routes. As used herein, the term “sequential” therapeutic use refers to administration of at least one additional agent beyond the one or more pulses of electricity (e.g., NSEPs) at different times, the administration route being identical or different. More particularly, sequential use refers to the whole administration of the additional agent before administration of the one or more pulses of electricity (e.g., NSEPs). It is thus possible to administer the additional agent over several minutes, hours, or days before applying the one or more pulses of electricity (e.g., NSEPs).

In one embodiment, the additional agent may include, for example, one or more antibiotic compound; one or more antimicrobial compound; one or more antibody; one or more biocidal agent; one or more nanoparticle; one or more self-assembling nanoparticle; one or more viral particle; one or more bacteriophage particle; one or more bacteriophage DNA; genetic material including but not limited to a plasmid, RNA, mRNA, siRNA, and an aptamer; one or more chemotherapy agent; one or more growth factor; one or more synthetic scaffold including but not limited to hydrogel and others; one or more natural scaffold including but not limited to collagen gel and decellularized tissue (whole, dissolved, denatured, or powdered); one or more electrode, one or more drug or pharmaceutical compound including but not limited to an anti-inflammatory agent, an inflammatory agent, a pain blocking agent, and a numbing agent; one or more microbes, and one or more bacteria.

If the additional agent is an antibiotic compound, such an antibiotic compound may include any of a number of different classes or types of antibiotics. Examples include aminoglycosides, ansamycins, carbapenems, cephalosporins, antibiotic glycopeptides, lincosamides, abitbiotic lipopeptides, macrolides, monobactams, nitrofurans, oxazolidinones, penicillins, quinolones, fluoroquinolones, sulfonamides, tetracyclines, or others. Any antibiotic from any of these categories may be used in accordance with aspects of the present disclosure. Non-limiting specific examples include, tobramycin, streptomycin, rifampicin, vancomycin, clindamycin, daptomycin, erythromycin, linezolid, penicillin, minocycline, pexiganan, fusidic acid, mupirocin, bacitracin, neomycin, polymixin B, and metronidazole. Other examples include metals or metal ions known to have antimicrobial or antibacterial effects, such as silver, copper, or zinc. In some examples, combinations of any two or more of the foregoing antibiotics or substances with antibiotic activity may be administered concurrently in accordance with an aspect of the present disclosure. In some examples, any one or more of the foregoing may also be explicitly excluded from use in accordance with an aspect of the present disclosure. Additional exemplary antibiotic agents include, but are not limited to, doxorubicin; actinomycin; aminoglycosides (e.g., neomycin, gentamicin, tobramycin); β-lactamase inhibitors (e.g., clavulanic acid, sulbactam); glycopeptides (e.g., vancomycin, teicoplanin, polymixin); ansamycins; bacitracin; carbacephem; carbapenems; cephalosporins (e.g., cefazolin, cefaclor, cefditoren, ceftobiprole, cefuroxime, cefotaxime, cefipeme, cefadroxil, cefoxitin, cefprozil, cefdinir); gramicidin; isoniazid; linezolid; macrolides (e.g., erythromycin, clarithromycin, azithromycin); mupirocin; penicillins (e.g., amoxicillin, ampicillin, cloxacillin, dicloxacillin, flucloxacillin, oxacillin, piperacillin); oxolinic acid; polypeptides (e.g., bacitracin, polymyxin B); quinolones (e.g., ciprofloxacin, nalidixic acid, enoxacin, gatifloxacin, levaquin, ofloxacin, etc.); sulfonamides (e.g., sulfasalazine, trimethoprim, trimethoprim-sulfamethoxazole (co-trimoxazole), sulfadiazine); tetracyclines (e.g., doxycyline, minocycline, tetracycline, etc.); monobactams such as aztreonam; chloramphenicol; lincomycin; clindamycin; ethambutol; mupirocin; metronidazole; pefloxacin; pyrazinamide; thiamphenicol; rifampicin; thiamphenicl; dapsone; clofazimine; quinupristin; metronidazole; linezolid; isoniazid; piracil; novobiocin; trimethoprim; fosfomycin; fusidic acid; or other topical antibiotics. Optionally, the antibiotic agents may also be antimicrobial peptides such as defensins, magainin and nisin; or lytic bacteriophage. The antibiotic agents can also be the combinations of any of the agents listed above.

In one embodiment, where the additional compound is an antimicrobial compound, the antimicrobial compound may include, for example, any agent that has the potential to reduce a microbe including but not limited to a fungus, such as Candida albicans, Candida auris, or species of Aspergillis. Various antifungal compounds may also be administered in accordance with an aspect of the present disclosure. Non-limiting examples include clotrimazole, econazole, miconazole, terbinafine, fluconazole, ketoconazole, and amphotericin, or other compounds known to have antifungal activities. In some embodiments, combinations of any two or more of the foregoing antifungals or substances with antifungal activity may be administered concurrently in accordance with an aspect of the present disclosure. In some examples, any one or more of the foregoing antifungals or substances with antifungal activity may also be explicitly excluded from use in accordance with an aspect of the present disclosure. In some other embodiments, one or more of the foregoing antibiotics or substances with antibiotic activity may be used in combination with any one or more of the foregoing antifungals or substances with antifungal activity in accordance with an aspect of the present disclosure.

In one embodiment, where the additional agent is an antibody, the antibody (“Ab”), which may also be call an immunoglobulin (“Ig”), may be any protein produced in a subject and use by the immune system to neutralize pathogens such as, for example, pathogenic bacteria and viruses.

In one embodiment, where the additional agent is a biocidal agent, the biocidal agent may be any substance or microorganism that is intended to destroy, deter, render harmless, or exert a controlling effect on any harmful organism. Biocidal agents may include, for example, preservatives, insecticides, disinfectants, and pesticides used for the control of organisms that are harmful to health or that cause damage to natural or manufactured products. The biocidal agent in some embodiments, may include, for example, a pesticide such as one or more of a fungicide, an herbicide, an insecticide, an algicide, a molluscicide, a miticide, a rodenticide, and a slimicide. The biocide may also include an antimicrobial biocide, including for example, a germicide, an antibiotic, an antibacterial, an antiviral, an antifungal, an antiprotozoal, and an antiparasite. In one embodiment, the biocide may be spermicide.

In one embodiment, the additional agent may be a nanoparticle, which includes but is not limited to any nanoparticle constructed with complex organic surface layers on a metal core such as gold or mineral core such as silica, as well as nanoparticles constructed with a polymeric organic core consisting of micelles, dendrimers, dextran, or PLGA. Nanoparticles are well known in the art.

Inhibiting the growth, proliferation, viability, reproduction, infectivity, or number of pathogens, for example pathogenic bacteria, viruses, and microbes, may be desirable. As used herein, reducing a number of viable pathogens includes any of the foregoing effects on pathogenic colonies or populations. Included are bacteriostatic and bactericidal effects. An antimicrobial composition, for example may be administered with application of NSEPs in accordance with the present disclosure with the result of increasing cell proliferation and differentiation and inhibiting the growth, proliferation, viability, reproduction, infectivity, or number of pathogens present, each and all of which are included in reducing a number of viable pathogens. A reduction in a number of viable pathogens (e.g., microbes) may result from a strictly bactericidal effect, a bacteriostatic effect, or a combination of the two.

A reduction in a number of viable pathogens may be identified by any of a number of known methods. For example, a treatment (i.e., applying of pulses of electricity in accordance with the methods disclosed herein) may be applied to one of two otherwise identical samples, then the samples cultured to measure pathogen growth following said applying pulses of electricity as compared to following absence of said applying pulses of electricity. If fewer pathogens are present after culturing the sample to which said treatment had been applied relative to the untreated sample, the treatment reduced a number of viable pathogens. A sample may be any surface, composition, liquid, substance, surface, tissue, or other material to which treatment as disclosed herein may be applied. In one embodiment, applying one or more pulses of electricity as disclosed herein and a beneficial composition (e.g., an antimicrobial composition) to a subject, such as a human or non-human animal subject, results in less infection (less in severity, less in duration, or both, or absence of infection) than results under similar circumstances, or than would have resulted, without treatment. For example, application of such treatment may slow growth of infectious pathogens (e.g., microbes) or otherwise render them more susceptible to a subject's immune system. Such examples of reduced infection are examples of reducing a number of viable pathogens and microbes.

In some examples, applying as disclosed herein may slow or prevent proliferation of pathogens such as microbes and thereby hasten a reduction in number of viable pathogens (e.g., increase susceptibility to a subject's immune system). In other examples, applying as disclosed herein may kill pathogens without immediately eliminating or removing them. Both are examples of a treatment reducing a number of viable pathogens.

In other examples, a reduction in a viable number of pathogens (e.g., microbes) might not result in a reduced duration, degree, or severity of an infection but may be evinced by culturing a sample and ascertaining an amount of pathogenic growth supported by such sample (following treatment as opposed to absent treatment). Other measures of a number of viable pathogens may be used as well, such as quantitative measures of microbial markers (antigens, genetic material, etc.) present in a sample, or microscopic or other known detection method. In some examples, such reduction of a number of viable pathogens may be evident within about 1 hr, about 2 hr, about 3 hr, about 4 hr, about 5 hr, about 6 hr, about 7 hr, about 8 hr, about 9 hr, about 10 hr, about 11 hr, about 12 hr, about 13 hr, about 14 hr, about 15 hr, about 16 hr, about 17 hr, about 18 hr, about 19 hr, about 20 hr, about 21 hr, about 22 hr, about 23 hr, about 24 hr, about 25 hr, about 26 hr, about 27 hr, about 28 hr, about 29, hr, about 30 hr, about 31 hr, about 32 hr, about 33 hr, about 34 hr, about 35 hr, about 36 hr, about 37 hr, about 38 hr, about 39 hr, about 40 hr, about 41 hr, about 42 hr, about 43 hr, about 44 hr, about 45 hr, about 46 hr, about 47 hr, about 48 hr, about 54 hr, about 60 hr, about 66 hr, about 72 hr, about 78 hr, about 84 hr, about 90 hr, about 96 hr, about 4.5 days, about 5 days, about 5.5 days, about 6 days, about 6.5 days, about 7 days, about 10 days, about 14 days, about 17 days, about 21 days, or about 28 days after applying one or more pulses of electricity. In this case, “about” means within +/−15% of the duration indicated.

If the additional agent administered is a bacteria, the bacteria may be, for example, a “probiotic”, which refers to any organism, particularly microorganisms that exert a beneficial effect on the host animal such as increased health or resistance to disease. Probiotic organisms can exhibit one or more of the following characteristics: non-pathogenic or non-toxic to the host; are present as viable cells, preferably in large numbers; microbicidal or microbistatic activity or effect toward pathogenic bacteria; enhanced urogenital tract health; capable of survival, metabolism, and persistence in the gut environment (e.g., resistance to gastrointestinal acids, secretions, and low pH); adherence to epithelial cells, particularly the epithelial cells of the gastrointestinal tract; anticarcinogenic activity; immune modulation activity, particularly immune enhancement; modulatory activity toward the endogenous flora; antiseptic activity in or around wounds and enhanced would healing; reduction in intestinal permeability; reduction in diarrhea; reduction in allergic reactions; reduction in neonatal necrotizing enterocolitis; and reduction in inflammatory bowel disease.

The probiotic cell may be, for example, Escherichia coli, Bifidobacterium longum, Bifidobacterium lactis, Bifidobacterium animalis, Bifidobacterium breve, Bifidobacterium infantis, Bifidobacterium adolescentis, Lactobacillus acidophilus, Lactobacillus casei, Lactobacillus paracasei, Lactobacillus salivarius, Lactobacillus reuteri, Lactobacillus rhamnosus, Lactobacillus johnsonii, Lactobacillus plantarum, Lactobacillus fermentum, Lactococcus lactis, Streptococcus thermophilus, Lactococcus lactis, Lactococcus diacetylactis, Lactococcus cremoris, Lactobacillus bulgaricus, Lactobacillus helveticus, Lactobacillus delbrueckii, or mixtures thereof.

An additional agent may be applied to a surface, solution, or substance, together with application of one or more pulses of electricity as disclosed herein, in order to reduce a number of viable pathogenic organisms on said surface or in such solution or substance, in accordance with the present disclosure. In one embodiment, an additional agent such as an antimicrobial may be applied or administered to a living subject such as a human and one or more pulses of electricity applied in accordance with the present disclosure. For example, an acute, chronic, sub-acute, sub-chronic, treatment-refractory, or other microbial infection, such as a bacterial or fungal infection, may be present in a subject such as a human subject. An additional agent such as an antimicrobial composition may be applied or administered to such subject and one or more pulses of electricity applied to reduce a number of viable microbes, such as to eliminate, remove, reduce, ameliorate, or otherwise treat such infection. In another example, such infection may be anticipated or a risk of such infection may be present, such as in an immunocompromised subject, or in conjunction with surgery or wound or trauma, or known or expected exposure to an infectious pathogen such as a microbe, whereupon an antimicrobial composition may be administered with application of one or more pulses of electricity prophylactically, to prevent development of infection or proliferation of an infectious seed of microbe that may be present or suspected of being present. Such examples are included with reducing an amount of viable pathogens as the term is used herein.

The additional agent may optionally be administered by any of various medically known or accepted or approved means of applying or administering such beneficial compositions. Examples include oral, parenteral (including subcutaneous, intradermal, intramuscular, intravenous and intraarticular), rectal and topical (including dermal, buccal, sublingual and intraocular) administration. An additional agent may be formulated as appropriate for such administration, which may be tailored to a given purpose, such as in a tablet, capsule, or other form for oral administration or injectable formulation for injection, or gel, cream, powder, ointment, or other composition for rectal or dermal application, etc. In some examples, one or more additional agent may be included in the surface of a material or an apparatus to be implanted on or within the body of a subject such as a human subject configured or otherwise formulated to have or promote an antimicrobial effect at the surface of such material or apparatus or to be released therefrom and have such an antimicrobial effect in tissue in the vicinity of such material or apparatus.

In accordance with an aspect of the present disclosure, one or more pulses of electricity may be administered to such a subject to enhance an effect of such beneficial composition. For example, a subject may receive or may have received systemic treatment with a beneficial composition such that application of one or more pulses of electricity to a part of the subject's body enhances a beneficial effect of said beneficial composition where one or more pulses of electricity are applied, reducing a number of viable pathogens. In another example, a beneficial composition may be topically applied, such as in a cream or ointment or powder or other form, or locally injected, or present in a material or apparatus implanted or to be implanted, and one or more pulses of electricity applied at a site of the beneficial composition thereby applied, to enhance effectiveness or otherwise reduce a number of viable pathogens there. Skilled persons would comprehend that various ways to apply beneficial compositions could be used in accordance with an aspect of the present disclosure.

In one embodiment, an additional agent comprising a beneficial composition or substance may be applied or present at a concentration, or to achieve a concentration locally, that alone does not have an effect on a number of viable pathogens (e.g., microbes) at a given site. In another embodiment, a beneficial composition or substance may be applied or present at a concentration, or to achieve a concentration locally, that alone has only low effect on a number of viable pathogens (e.g., microbes) at a given site. In either example, in accordance with an aspect of the present disclosure, applying one or more pulses of electricity to cells may increase a reduction in a number of viable pathogens otherwise resulting from application of the beneficial composition in the absence of one or more pulses of electricity. A beneficial composition may be administered at a concentration that is not effective at all or only minimally effective at reducing a number of viable pathogens of a given species or strain when applied in the absence of one or more pulses of electricity, whereas combining such administration with application of one or more pulses of electricity cause an increase in reduction of viable pathogens. In another embodiment, one or more pulses of electricity may be ineffective or only marginally effective or their own in reducing a number of viable pathogens on their own but rendered effective in the presence of an agent including a beneficial composition.

In one embodiment, a time frame required for effectiveness of a beneficial composition in reducing a number of viable pathogens may be reduced when administered in combination with application of one or more pulses of electricity. Conventionally, an antimicrobial composition such as an antibiotic or antifungal may require hours, days, or even weeks to be effective in reducing a number of viable microbes, or to be fully effective in preventing or eliminating an infection. Thus, whereas a given concentration of an antimicrobial composition may be effective in reducing a number of viable microbes on its own, combining its administration with application of one or more pulses of electricity as disclosed herein may result in reduction of a number of viable pathogens following a shorter time span of exposure to the beneficial composition at that concentration than would otherwise be required before such an effect of the beneficial composition results.

Any suitable approach for delivery of the additional agents can be utilized to practice this aspect. Typically, the agent will be administered to a patient in a vehicle that delivers the agent(s) to the target cell, tissue, or organ. Exemplary routes of administration include, without limitation, by intratracheal inoculation, aspiration, airway instillation, aerosolization, nebulization, intranasal instillation, oral or nasogastric instillation, intraperitoneal injection, intravascular injection, topically, transdermally, parenterally, subcutaneously, intravenous injection, intra-arterial injection (such as via the pulmonary artery), intramuscular injection, intrapleural instillation, intraventricularly, intralesionally, by application to mucous membranes (such as that of the nose, throat, bronchial tubes, genitals, and/or anus), or implantation of a sustained release vehicle.

In some embodiments, an additional agent is administered orally, topically, intranasally, intraperitoneally, intravenously, subcutaneously, or by aerosol inhalation. In some embodiments, an additional agent is administered via aerosol inhalation. In some embodiments, an additional agent can be incorporated into pharmaceutical compositions suitable for administration, as described herein.

The amount to be administered will, of course, vary depending upon the treatment regimen. Generally, an agent is administered to achieve an amount effective for cell differentiation or stimulation, or treatment of the condition causing or making a subject susceptible to having reduced differentiation or stimulation of cells. Thus, a therapeutically effective amount can be an amount which is capable of at least partially treating or preventing such a condition. This includes, without limitation, delaying the onset of infection. The dose required to obtain an effective amount may vary depending on the agent, formulation, and individual to whom the agent is administered.

Dosage, toxicity and therapeutic efficacy of the agents or compositions of the present invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit high therapeutic indices may be desirable. While compositions that exhibit toxic side effects may be used, care should be taken to design a delivery system that targets such compositions to the site of affected tissue in order to minimize potential damage to uninfected cells and, thereby, reduce side effects.

A third aspect relates to a method of regenerating cells. The method includes applying one or more pulses of electricity to cells, each pulse of electricity having a duration of between about 10 nanoseconds and about 1,000 nanoseconds, wherein said pulses of electricity are applied under conditions effective to promote cell regeneration.

This aspect is carried out in accordance with the previously described aspects under conditions effective to regenerate cells.

A fourth aspect relates to a method of promoting nodule formation. The method includes applying one or more pulses of electricity to cells, each pulse of electricity having a duration of between about 10 nanoseconds and about 1,000 nanoseconds, wherein said pulses of electricity are applied under conditions effective to promote nodule formation.

This aspect is carried out in accordance with the previously described aspects under conditions effective to promote nodule formation. In one embodiment, promoting nodule formation includes bone formation.

A fifth aspect relates to a method of promoting myotube formation. The method includes applying one or more pulses of electricity to cells, each pulse of electricity having a duration of between about 10 nanoseconds and about 1,000 nanoseconds, wherein said pulses of electricity are applied under conditions effective to promote myotube formation.

This aspect is carried out in accordance with the previously described aspects under conditions effective to promote myotube formation.

The present disclosure may be further illustrated by reference to the following examples.

### EXAMPLES

### Example 1—Materials and Methods

Isolation and culture of Primary myoblasts—Primary myoblasts are isolated from hind limb skeletal muscle of 4-week mice (PMID: 27880908). Briefly, muscles are minced and digested in type B collagenase and dispase II mixture (Roche). Digested cells are harvested and cultured in growth media, F-10 Ham's medium (Thermo Fisher Scientific) supplemented with 20% fetal bovine serum (FBS, Atlanta), 4 ng/ml basic fibroblast growth factor (Thermo Fisher Scientific) and 1% penicillin-streptomycin (Thermo Fisher Scientific) on collagen-coated dishes. Primary myoblasts are isolated and purified after pre-plating two to three times. Primary myoblasts are then induced to differentiate by growing in Dulbecco's Modified Eagle Medium (DMEM, Sigma) supplemented with 2% horse serum (Sigma) for at least two days.

Culture of primary human osteoblasts—Primary human osteoblasts obtained from vertebrae (Sciencell®) are cultured in DMEM/F-12 media (Gibco) supplemented with 10% FBS with 1% L-Glutamine+1% Penicillin-streptomycin antibiotic in tissue culture grade flasks. Cells are removed from the adhered surface and concentrated to 2×10⁶ cells/ml prior to pulsing in a 2 mm gap cuvette. These are then plated in 96 well plates with a methyl thiazolyl tetrazolium (MTT) stain to take counts 4 h, 24 h, and 48 hours after NSEP treatment, followed by plating cells at 1×10⁴, 2.5×10⁴ and 5×10⁴ cells/well in a 24 well plate for immunostaining prior to fluorescence studies.

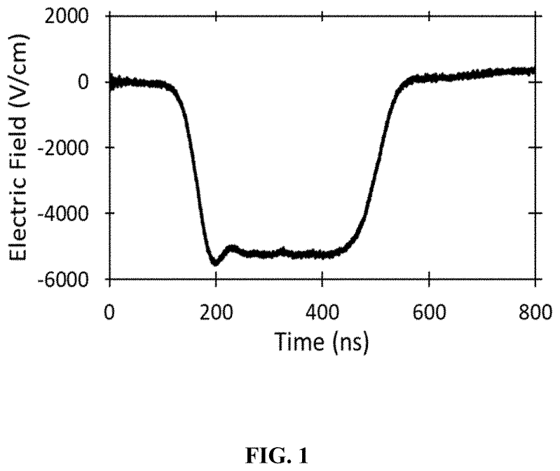

Nanosecond electric pulse exposure—To maintain consistent stem cell regenerative capacity, myoblasts between the second and eighth passage are used for all experiments. Myoblasts are cultured in 10 cm dishes until achieving 80% confluency. Similarly, osteoblasts are passaged for pulsing upon reaching 80% confluency. Both samples are diluted to a concentration of 2×10⁶ cells/ml, placed in standard 2 mm electroporation cuvettes (Dot Scientific®), and 300 ns EPs using a pulse generator consisting of 24 capacitors and inductors arranged as a standard Blumlein circuit design. The pulse generator is powered by an EJ series Glassman® high voltage 600 W DC power supply, and activated with a spark gap switch to produce EPs of 300 ns duration at the peak with rise and fall times of approximately 30 ns. Each treatment exposed the samples to five pulses at a repetition frequency of 1 Hz. The resistance of the samples in the cuvettes matched the pulse generator's impedance to prevent pulse reflection. The applied voltage across the cuvette is measured using a LeCroy PPE 20 kV high voltage probe with a 1000:1 attenuation that feeds into a TeleDyne LeCroy® Waverunner 6 Zi Oscilloscope capable of measuring up to 4 GHz. FIG. 1 shows a typical measured waveform. The electric field across the parallel plates is reported as E=V/d, where Vis the peak voltage of the applied pulse in kV and d is the gap distance in cm (0.2 cm here).

Plating after electric pulse treatment—Immediately after treating the myoblasts with five pulses, a hemocytometer is used to determine the number of surviving cells and the viability using trypan blue staining to ensure that the same number of live cells is plated in each well. This requires diluting the samples and accounting for cell death to ensure the plating of 2×10⁴ live cells per well. Three wells each are used for MTT assays at 24 and 48 hours after pulsing in 96 well plates.

Similarly, osteoblasts are treated with five pulses, but plated at the same volume of cell solution, using the control as a reference. The control (unpulsed) sample contained 2×10⁴ live cells in 10 μL of fluid. The same volume is plated for all treated samples to better simulate clinical conditions/applications. The cells are plated in three wells for each condition in 24 well plates in a total volume of 200 μL. Counts are taken 4 h, 24 h, and 48 hours after plating with 4 hours selected as the initial time to ensure sufficient time for the cells to adhere to the cell well surface.

Cell proliferation assay—Cell proliferation is assessed using the MTT cell proliferation assay kit from ATCC (ATCC 30-1010K). Experiments consist of adding 10 μl of 5 mg/ml MTT to each well of the 96 well plates containing pulsed cells at 0 h, 24 h, and 48 hours after treatment. The media is drained 4 hours after incubation at 37° C. for the initial (0 h) count to allow adherence to the dish surface. Purple formazan dyes are dissolved in 100 μl DMSO in each well and absorbance is measured at 570 nm for the myoblast experiments.

For osteoblasts, the media is drained 1 hour prior to counting and a mixture of 100 μL media and 20 μL MTT is added to each well, which is allowed to stain for 1 h. Next, 100 μL of the stained solution is transferred from each well to a 96 well plate to count with a photospectrometer at a wavelength of 570 nm.

Immunofluoresence—Pulsed myoblast cells are seeded in 24-well plates of 15.6 mm diameter and 3.4 mL volume at a density of 3×10⁵ cells/well. After 48 h, the cells are cultured in growth media or differentiation media for 72 h. After removing the media, the cells are fixed in 4% paraformaldehyde (PFA) for 5 min and incubated in 100 mM glycine for 15 min. Cells are then permeabilized in blocking buffer containing 5% goat serum, 2% bovine serum albumin, 0.2% Triton X-100, and 0.1% sodium azide in PBS for 1 h. Myosin heavy chain protein is used as the maturation marker of myoblasts. The primary antibody MF20 (R&D Systems®, #MAB4470, mouse) is added to the blocking buffer in a 1:30 dilution and applied to cells overnight at 4° C. Cells are then incubated in an anti-mouse IgG2b 568 (Invitrogen) secondary antibody for 1 hour and cell nuclei are co-stained with 1 μM DAPI. Between four and six fluorescent images are captured per well with a CoolSnap HQ charge coupled-device camera (Photometrics) and a Leica DM6000 microscope.

Osteoblast staining—Osteoblasts are fixed in 0.5% glutaraldehyde solution in phosphate buffered saline for one hour. Cultures are rinsed with deionized water and stained with Alizarin Red stain, 40 mM in deionized water pH 4.2 (Sigma A5533). Stain is placed on cultures for one hour with agitation. Cultures are destained with repeated deionized water rinses for 24 h.

### Example 2—EPs Increased Proliferation and Differentiation in Myoblasts

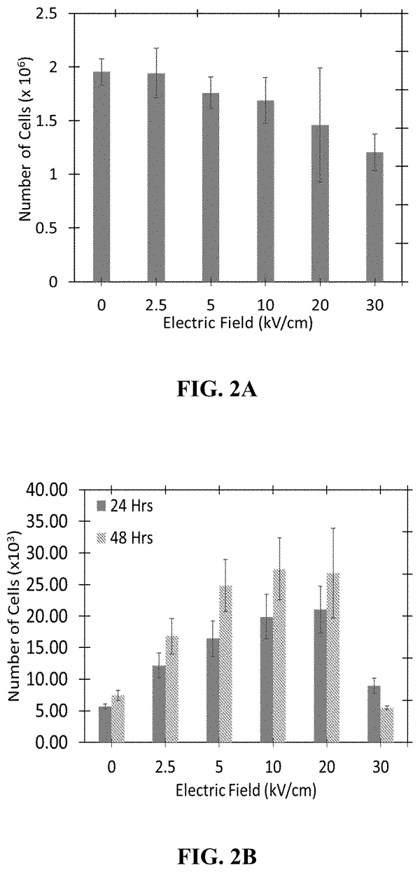

Myoblasts were treated with five 300 ns EPs at 0, 2.5, 5, 10, 20, or 30 kV/cm. FIG. 2A shows that the number of cells was unchanged at 2.5 kV/cm and decreased with increasing EP field strength from 5-30 kV/cm. FIG. 2A shows that the number of myoblast cells that survive EP treatment decreased for increased field strengths. A fixed volume of cells was then seeded to monitor the growth rate. FIG. 2 B shows that the 2.5 kV/cm EPs increase myoblast proliferation by twofold without impairing survival, while the 5, 10, and 20 kV/cm EPs increase growth rate by three- to four-fold with a reduced survival rate. The growth rate was lower 48 hours post-treatment at 30 kV/cm, suggesting that these EPs may exceed a threshold for damaging myoblast physiology. The increased proliferation resulted in a high cell density that caused the myoblasts to differentiate spontaneously without the serum withdrawal typically used to induce myoblast differentiation.

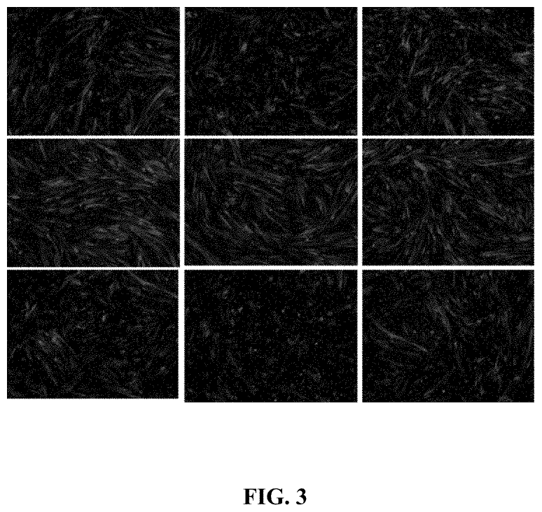

Myotube differentiation is the physiological process for myoblast maturation. Differentiation was studied two days after EP treatment to assess the impact on myoblast function. FIG. 3 shows that replacing the growth media with differentiation media causes more fused myoblasts for 5 kV/cm EPs compared to control, as indicated by the presence of the myosin heavy chains (stained in red), the maturation marker in the myotubes. In contrast, fewer fused myoblasts formed at 20, 25 and 30 kV/cm, indicating that lower intensity EPs (2.5-5 kV/cm) maintained myoblast maturation while higher intensity EPs may impair myoblast differentiation. The initial plated myoblast cells showed an increase in the myosin heavy chains (stained in red) due to fusion with other cells to form multinucleated myotubes (blue highlights the nuclei of the cells).

Combined, FIGS. 2A-2B show that EP treatment increased cell proliferation compared to the untreated control with each image taken from a different well of a 24-well cell culture dish and FIG. 3 shows that this increases myoblast differentiation at low EP intensity (5 kV/cm) but not at high EP intensity (25 kV/cm).

### Example 3—EPs Increased Proliferation and Differentiation in Osteoblasts

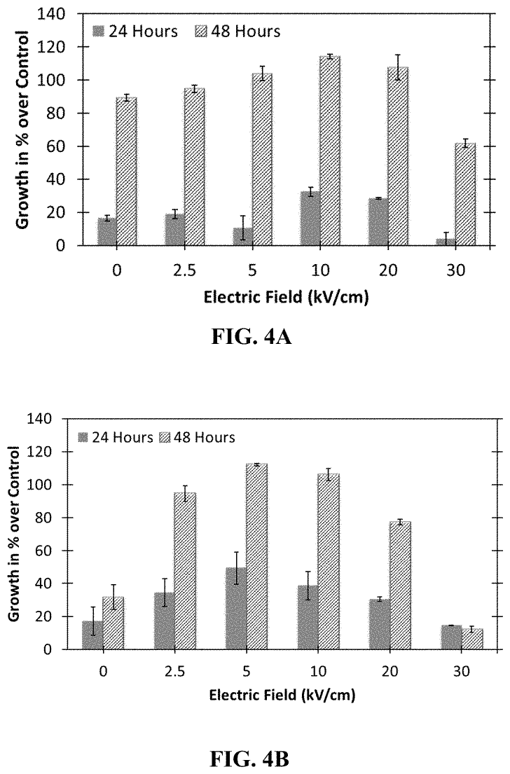

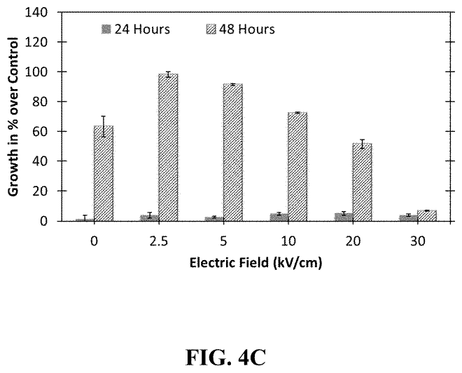

Similarly, osteoblast concentration was set to 2×10⁶ cells/ml prior to pulsing. For the untreated control sample, 10 μL of this sample corresponds to 20,000 cells/well. This same volume of fluid was then plated for all samples in 24 well plates (2 cm²/well). The initial population count was taken using an automated cell counter (Countess®). MTT assays were performed at 4 h, 24 h, and 48 hours (n=3). The growth curves for pulsed osteoblasts measured from the MTT assay are reported as percentage of growth compared to the untreated control at 24 hours and 48 hours after treatment. FIGS. 4A-4C summarize the results from three identical tests with the exception of the initial osteoblast concentration, which impacts the growth curves after NSEP exposure. Thus, rather than averaging the results and obtaining large error bars that would hide the general NSEP behavior, the individual results and observed general trends are reported.

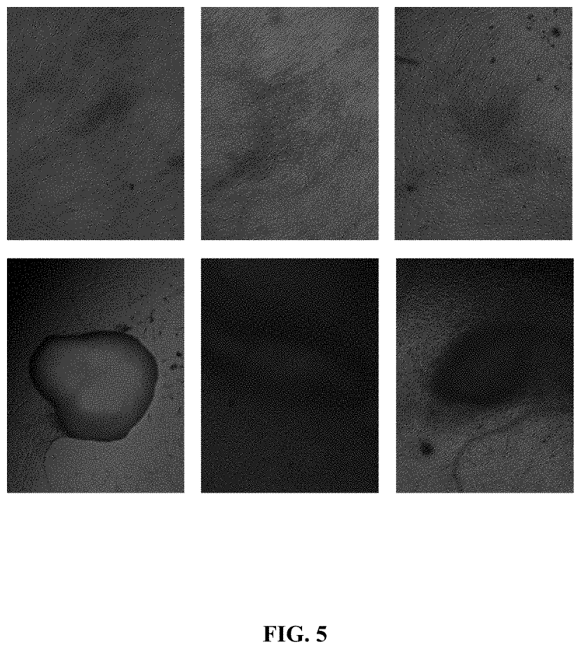

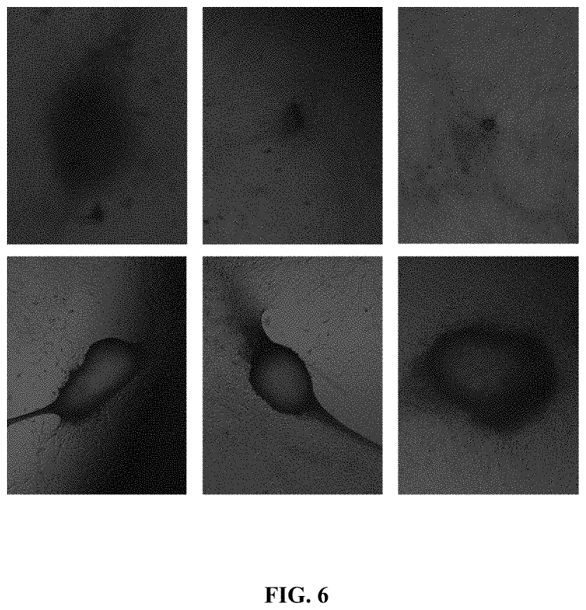

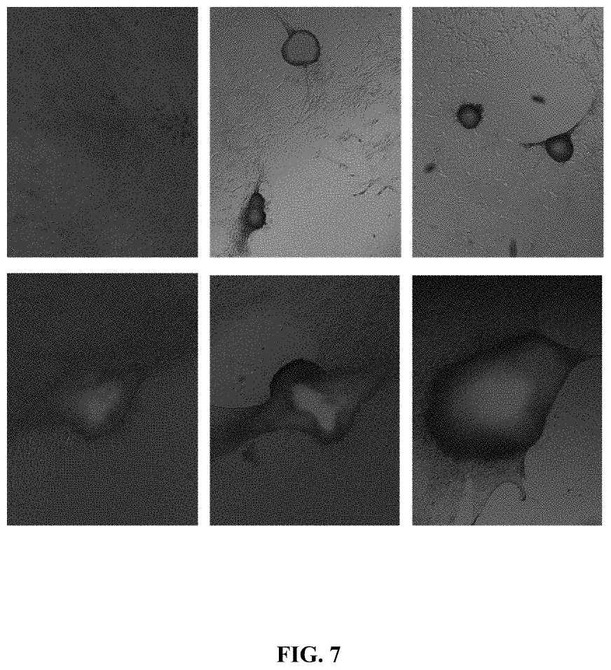

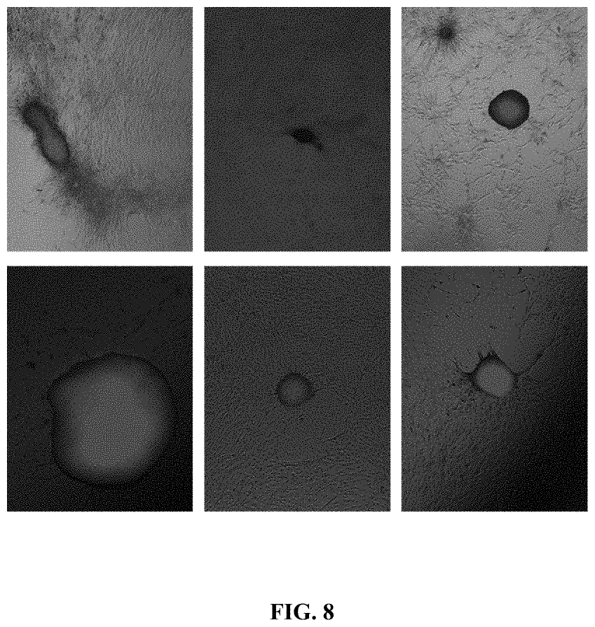

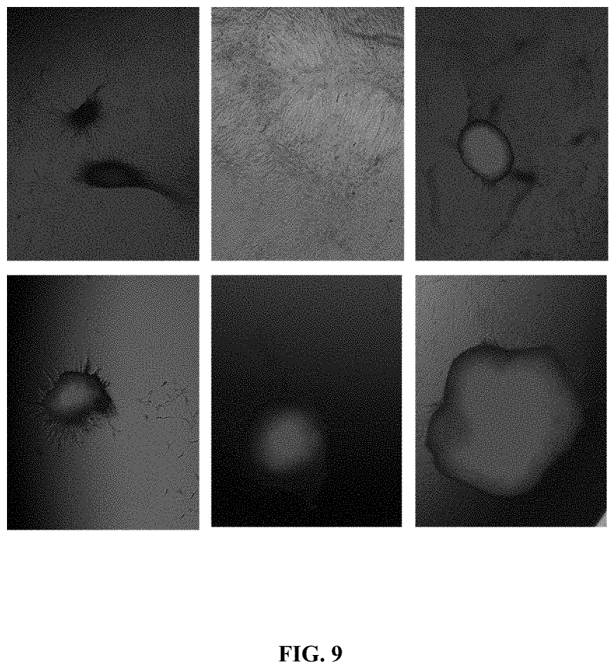

As with the myoblasts, the osteoblasts were plated, stained, and photographed 7 days and 14 days after plating, as shown in FIGS. 5-9 for the untreated control, and cells were exposed to five 300 ns EPs at 1.5 kV/cm, 2.5 kV/cm, 5 kV/cm, and 10 kV/cm, respectively. Each figure shows representative images from each of the three wells with the red color representing nodule formation, which indicates bone formation. While intensity of the red color is controlled for, larger red spots indicate greater nodule formation. All field strengths induced noticeably increased nodule formation 14 days after treatment with treatments of 2.5 kV/cm and higher inducing some nodule formation even after 7 days. Table 1 reports the nodule counts 14 days after exposing the osteoblasts to five 300 ns EPs with various intensities, as well as the unpulsed control. While the variation is sufficient that the results have no statistical significance, it is noted that the intermediate pulse durations of 1.5 kV/cm and 2.5 kV/cm generally lead to the largest increase in nodule formation for each replicate in each trial. No nodules form in the control and only a single nodule forms following the 2.5 kV/cm treatment in one of the replicates in Trial 1. In Trial 2, the 2.5 kV/cm treatment increased nodule formation compared to control in three of the four replicates and by 63.6% compared to control on average, compared to 36.4% and 54.5% for the 3 kV/cm and 5 kV/cm treatments, respectively. While noticeable increases in nodule formation in Trial 3 with the 1.5 kV/cm were observed, all EP trains below 10 kV/cm were effective for this trial. As a whole, this data suggests that the 1.5 to 5 kV/cm EP trains are generally effective while nodule formation declines for the 10 kV/cm trains.

TABLE 1
Nodule growth for each replicate of three trials
of osteoblasts 14 days after exposure to five 300
ns electric pulses of various electric field intensities.

| Field | | | | | |
|---|---|---|---|---|---|
| (kV/cm) | 0 | 1.5 | 2.5 | 5 | 10 |
| TRIAL | 0 | 0 | 0 | 0 | 0 |
| 1 | | | | | |
| | 0 | 0 | 1 | 0 | 0 |
| | 0 | 0 | 0 | 0 | 0 |
| Average | 0.0 | 0.0 | 0.3 | 0.0 | 0.0 |
| TRIAL | 2 | 6 | 7 | 7 | 2 |
| 2 | | | | | |
| | 2 | 4 | 3 | 6 | 3 |
| | 5 | 3 | 2 | 2 | 3 |
| | 2 | 5 | 3 | 2 | 4 |
| Average | 2.8 | 4.5 | 3.8 | 4.3 | 3.0 |
| TRIAL | 2 | 2 | 5 | 2 | 1 |
| 3 | | | | | |
| | 0 | 3 | 1 | 1 | 0 |
| | 0 | 2 | 1 | 1 | 1 |
| Average | 0.7 | 2.3 | 2.3 | 1.3 | 0.7 |

### Example 4—EPs Induce Proliferation, Differentiation, Maturation, and Nodule Formation

The results presented herein indicate that applying five 300 ns EPs of appropriate electric field intensity to either myoblasts or osteoblasts can induce proliferation and myotube maturation or nodule formation, respectively. For myoblasts, electric fields from 2.5 kV/cm to 20 kV/cm increased myoblast population compared to untreated control 24 hours and 48 hours after treatment while electric fields above 2.5 kV/cm resulted in reduced cell population immediately after treatment. The cell population growth does not differ statistically significantly from the control sample. Immunostaining indicated that an applied electric field of 5 kV/cm increased myotube formation compared to either untreated control or myoblasts exposed to 25 kV/cm. Thus, an optimal field intensity could selectively enhance myotube formation. Similar results were observed for the osteoblasts, including nodule formation within 48 to 72 hours and increased nodule formation for higher field strengths.

Thus, the immunostaining images revealed increased proliferation after pulsing either cell type, which could contribute to increased cell differentiation. Prior research showed that electric field induced ion movement could create currents that affected transmembrane voltage, which can determine the differentiation pathway of mesenchymal stem cells. The release of intracellular stores of Ca′ can also affect growth kinetics. Research shows that electrical stimulation can enhance osteoblast differentiation by altering the transmembrane potential, which subsequently influences growth and differentiation. Since NSEPs target the plasma membrane, intracellular organelle membranes, intracellular calcium stores, and the cytoskeleton, it is likely that a similar release of stored intracellular ions and the inhibition or activation of other signaling pathways stimulated population growth and differentiation.

Future tests combining differentiating and non-differentiating media with NSEPs can determine whether this synergistically increases differentiation, analogous to past studies assessing the synergy of antimicrobial agents with NSEPs. Polymerase chain reaction (PCR) tests can analyze changes in mRNA and the transcriptome that could indicate whether NSEPs induce differentiation. Moreover, the current study focuses on just the impact of applied pulse energy by controlling the applied electric field; however, one could also vary the pulse duration, number of pulses, and even the delivery mechanism from conductive coupling to capacitive coupling. Future studies will involve a more detailed parametric study of pulse parameters, which will impact cellular target and intensity, while also further assessing the impact on membrane potential and calcium release. Ultimately, animal studies can demonstrate the potential utility of this approach for clinical applications in wound healing.

While the novel technology has been illustrated and described in detail in the figures and foregoing description, the same is to be considered as illustrative and not restrictive in character, it being understood that only the preferred embodiments have been shown and described and that all changes and modifications that come within the spirit of the novel technology are desired to be protected. As well, while the novel technology was illustrated using specific examples, theoretical arguments, accounts, and illustrations, these illustrations and the accompanying discussion should by no means be interpreted as limiting the technology. All patents, patent applications, and references to texts, scientific treatises, publications, and the like referenced in this application are incorporated herein by reference in their entirety.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A method of stimulating cell proliferation, said method comprising:
applying one or more pulses of electricity to cells, each pulse of electricity having a duration of between about 10 nanoseconds and about 1,000 nanoseconds, wherein said pulses of electricity are applied under conditions effective to stimulate cell proliferation.

2. The method of claim 1, wherein each pulse of electricity has a duration of between about 10 nanoseconds and about 300 nanoseconds.

3. The method of claim 1, wherein each pulse of electricity has a frequency of repetition in a range of between about 0.01 Hz to about 1,000 Hz.

4. The method of claim 3, wherein each pulse of electricity has a frequency of repetition in a range of between about 0.1 Hz to about 300 Hz.

5. The method of claim 4, wherein each pulse of electricity has a frequency of repetition in a range of between about 0.5 Hz to about 10 Hz.

6. The method of claim 1, wherein each pulse of electricity has an intensity peak in a range of about 1.0 kV/cm to about 30.0 kV/cm.

7. The method of claim 1, wherein each pulse of electricity has an intensity peak in a range of about 1.0 kV/cm and about 25.0 kV/cm.

8. The method of claim 7, wherein each pulse of electricity has an intensity peak in a range of about 5.0 kV/cm and about 10.0 kV/cm.

9. The method of claim 1, wherein each pulse of electricity has an intensity peak of about 1.0 kV/cm.

10. The method of claim 1, wherein each pulse of electricity has an intensity peak in a range of about 2.5 kV/cm to about 25.0 kV/cm.

11. The method of claim 1, wherein a 30 nanosecond rise and fall time is present between a peak intensity and a baseline intensity.

12. The method of claim 1, wherein a time between rise and fall times of a peak intensity and a baseline intensity is less than about 10 nanoseconds.

13. The method of claim 1, wherein said electrical pulses are applied to said cells in vivo.

14. The method of claim 1, wherein said electrical pulses are applied to said cells in vitro.

15. The method of claim 14 further comprising:
inserting said cells into a subject following applying of said one or more electrical pulses.

16. The method of claim 1, wherein said cells are selected from the group consisting of stem cells, satellite cells, myoblasts, osteoblasts, chondrocytes, fibroblasts, tenocytes, precursor cells, embryological cells, progenitor cells, mesenchymal stem cells, neural stem cells, glial progenitor cells, angioblast hematopoietic stem cells, induced pluripotent stem cells, allograft stem cells, and xenograft stem cells.

17. The method of claim 1, wherein said cells are subject to up to 150 pulses of electricity.

18. The method of claim 17, wherein said cells are subject to five or fewer pulses of electricity.

19. The method of claim 1 further comprising:
administering an additional agent.

20. The method of claim 19, wherein said additional agent is selected from group consisting of an antibiotic compound, an antimicrobial compound, an antibody, a biocidal agent, nanoparticles, self-assembling nanoparticles, viral particles, bacteriophage particles, bacteriophage DNA, genetic material, chemotherapy agent, growth factor, synthetic scaffold, natural scaffold, electrode, drug, a microbe, and a bacteria.

21. A method of promoting differentiation of cells, said method comprising:
applying one or more pulses of electricity to cells, each pulse of electricity having a duration of between about 10 nanoseconds and about 1,000 nanoseconds, wherein said pulses of electricity are applied under conditions effective to promote differentiation of cells.

22. The method of claim 21, wherein each pulse of electricity has a duration of between about 10 nanoseconds and about 300 nanoseconds.

23. The method of claim 21, wherein each pulse of electricity has a frequency of repetition in a range of between about 0.01 Hz to about 1,000 Hz.

24. The method of claim 23, wherein each pulse of electricity has a frequency of repetition in a range of between about 0.1 Hz to about 300 Hz.

25. The method of claim 24, wherein each pulse of electricity has a frequency of repetition in a range of between about 0.5 Hz to about 10 Hz.

26. The method of claim 21, wherein each pulse of electricity has an intensity peak in a range of about 1.0 kV/cm to about 30.0 kV/cm.

27. The method of claim 21, wherein each pulse of electricity has an intensity peak in a range of about 1.0 kV/cm and about 25.0 kV/cm.

28. The method of claim 27, wherein each pulse of electricity has an intensity peak in a range of about 5.0 kV/cm and about 10.0 kV/cm.

29. The method of claim 21, wherein each pulse of electricity has an intensity peak of about 1.0 kV/cm.

30. The method of claim 21, wherein each pulse of electricity has an intensity peak in a range of about 2.5 kV/cm to about 25.0 kV/cm.

31. The method of claim 21, wherein a 30 nanosecond rise and fall time is present between a peak intensity and a baseline intensity.

32. The method of claim 21, wherein a time between rise and fall times of a peak intensity and a baseline intensity is less than about 10 nanoseconds.

33. The method of claim 21, wherein said electric pulses are applied to said cells in vivo.

34. The method of claim 21, wherein said electrical pulses are applied to said cells in vitro.

35. The method of claim 34 further comprising:
inserting said cells into a subject following applying of said one or more electrical pulses.

36. The method of claim 21, wherein said cells are selected from the group consisting of stem cells, satellite cells, myoblasts, osteoblasts, chondrocytes, fibroblasts, tenocytes, precursor cells, embryological cells, progenitor cells, mesenchymal stem cells, neural stem cells, glial progenitor cells, angioblast hematopoietic stem cells, induced pluripotent stem cells, allograft stem cells, and xenograft stem cells.

37. The method of claim 21, wherein said cells are subject to up to 150 pulses of electricity.

38. The method of claim 37, wherein said cells are subject to five or fewer pulses of electricity.

39. The method of claim 21 further comprising:
administering an additional agent.

40. The method of claim 39, wherein said additional agent is selected from group consisting of an antibiotic compound, an antimicrobial compound, an antibody, a biocidal agent, nanoparticles, self-assembling nanoparticles, viral particles, bacteriophage particles, bacteriophage DNA, genetic material, chemotherapy agent, growth factor, synthetic scaffold, natural scaffold, electrode, drug, a microbe, and a bacteria.

41. A method of regenerating cells, said method comprising:
applying one or more pulses of electricity to cells, each pulse of electricity having a duration of between about 10 nanoseconds and about 1,000 nanoseconds, wherein said pulses of electricity are applied under conditions effective to promote cell regeneration.

42. The method of claim 41, wherein each pulse of electricity has a duration of between about 10 nanoseconds and about 300 nanoseconds.

43. The method of claim 41, wherein each pulse of electricity has a frequency of repetition in a range of between about 0.01 Hz to about 1,000 Hz.

44. The method of claim 43, wherein each pulse of electricity has a frequency of repetition in a range of between about 0.1 Hz to about 300 Hz.

45. The method of claim 44, wherein each pulse of electricity has a frequency of repetition in a range of between about 0.5 Hz to about 10 Hz.

46. The method of claim 41, wherein each pulse of electricity has an intensity peak in a range of about 1.0 kV/cm to about 30.0 kV/cm.

47. The method of claim 44, wherein each pulse of electricity has an intensity peak in a range of about 1.0 kV/cm and about 25.0 kV/cm.

48. The method of claim 47, wherein each pulse of electricity has an intensity peak in a range of about 5.0 kV/cm and about 10.0 kV/cm.

49. The method of claim 41, wherein each pulse of electricity has an intensity peak of about 1.0 kV/cm.

50. The method of claim 41, wherein each pulse of electricity has an intensity peak in a range of about 2.5 kV/cm to about 25.0 kV/cm.

51. The method of claim 41, wherein a 30 nanosecond rise and fall time is present between a peak intensity and a baseline intensity.

52. The method of claim 41, wherein a time between rise and fall times of a peak intensity and a baseline intensity is less than about 10 nanoseconds.

53. The method of claim 41, wherein said electric pulses are applied to said cells in vivo.

54. The method of claim 41, wherein said electrical pulses are applied to said cells in vitro.

55. The method of claim 54 further comprising:
inserting said cells into said subject following applying of said one or more electrical pulses.

56. The method of claim 41, wherein said cells are selected from the group consisting of stem cells, satellite cells, myoblasts, osteoblasts, chondrocytes, fibroblasts, tenocytes, precursor cells, embryological cells, progenitor cells, mesenchymal stem cells, neural stem cells, glial progenitor cells, angioblast hematopoietic stem cells, induced pluripotent stem cells, allograft stem cells, and xenograft stem cells.

57. The method of claim 41, wherein said cells are subject to up to 150 pulses of electricity.

58. The method of claim 57, wherein said cells are subject to five or fewer pulses of electricity.

59. The method of claim 41 further comprising:
administering an additional agent.

60. The method of claim 59, wherein said additional agent is selected from group consisting of an antibiotic compound, an antimicrobial compound, an antibody, a biocidal agent, nanoparticles, self-assembling nanoparticles, viral particles, bacteriophage particles, bacteriophage DNA, genetic material, chemotherapy agent, growth factor, synthetic scaffold, natural scaffold, electrode, drug, a microbe, and a bacteria.

61. A method of promoting nodule formation, said method comprising:
applying one or more pulses of electricity to cells, each pulse of electricity having a duration of between about 10 nanoseconds and about 1,000 nanoseconds, wherein said pulses of electricity are applied under conditions effective to promote nodule formation.

62. The method of claim 61, wherein each pulse of electricity has a duration of between about 10 nanoseconds and about 300 nanoseconds.

63. The method of claim 61, wherein each pulse of electricity has a frequency of repetition in a range of between about 0.01 Hz to about 1,000 Hz.

64. The method of claim 63, wherein each pulse of electricity has a frequency of repetition in a range of between about 0.1 Hz to about 300 Hz.

65. The method of claim 64, wherein each pulse of electricity has a frequency of repetition in a range of between about 0.5 Hz to about 10 Hz.

66. The method of claim 61, wherein each pulse of electricity has an intensity peak in a range of about 1.0 kV/cm to about 30.0 kV/cm.

67. The method of claim 61, wherein each pulse of electricity has an intensity peak in a range of about 1.0 kV/cm and about 25.0 kV/cm.

68. The method of claim 67, wherein each pulse of electricity has an intensity peak in a range of about 5.0 kV/cm and about 10.0 kV/cm.

69. The method of claim 61, wherein each pulse of electricity has an intensity peak of about 1.0 kV/cm.

70. The method of claim 61, wherein each pulse of electricity has an intensity peak in a range of about 2.5 kV/cm to about 25.0 kV/cm.

71. The method of claim 61, wherein a 30 nanosecond rise and fall time is present between a peak intensity and a baseline intensity.

72. The method of claim 61, wherein a time between rise and fall times of a peak intensity and a baseline intensity is less than about 10 nanoseconds.

73. The method of claim 61, wherein said electric pulses are applied to said cells in vivo.

74. The method of claim 61, wherein said electrical pulses are applied to said cells in vitro.

75. The method of claim 74 further comprising:
inserting said cells into a subject following applying of said one or more electrical pulses.

76. The method of claim 61, wherein said cells are osteoblasts.

77. The method of claim 61, wherein said cells are subject to up to 150 pulses of electricity.

78. The method of claim 77, wherein said cells are subject to five or fewer pulses of electricity.

79. The method of claim 61 further comprising:
administering an additional agent.

80. The method of claim 79, wherein said additional agent is selected from group consisting of an antibiotic compound, an antimicrobial compound, an antibody, a biocidal agent, nanoparticles, self-assembling nanoparticles, viral particles, bacteriophage particles, bacteriophage DNA, genetic material, chemotherapy agent, growth factor, synthetic scaffold, natural scaffold, electrode, drug, a microbe, and a bacteria.

81. The method of claim 61, wherein promoting nodule formation comprises bone formation.

82. A method of promoting myotube formation, said method comprising:
applying one or more pulses of electricity to cells, each pulse of electricity having a duration of between about 10 nanoseconds and about 1,000 nanoseconds, wherein said pulses of electricity are applied under conditions effective to promote myotube formation.

83. The method of claim 82, wherein each pulse of electricity has a duration of between about 10 nanoseconds and about 300 nanoseconds.

84. The method of claim 82, wherein each pulse of electricity has a frequency of repetition in a range of between about 0.01 Hz to about 1,000 Hz.

85. The method of claim 84, wherein each pulse of electricity has a frequency of repetition in a range of between about 0.1 Hz to about 300 Hz.

86. The method of claim 85, wherein each pulse of electricity has a frequency of repetition in a range of between about 0.5 Hz to about 10 Hz.

87. The method of claim 82, wherein each pulse of electricity has an intensity peak in a range of about 1.0 kV/cm to about 30.0 kV/cm.

88. The method of claim 82, wherein each pulse of electricity has an intensity peak in a range of about 1.0 kV/cm and about 25.0 kV/cm.

89. The method of claim 88, wherein each pulse of electricity has an intensity peak in a range of about 5.0 kV/cm and about 10.0 kV/cm.

90. The method of claim 82, wherein each pulse of electricity has an intensity peak of about 1.0 kV/cm.

91. The method of claim 82, wherein each pulse of electricity has an intensity peak in a range of about 2.5 kV/cm to about 25.0 kV/cm.

92. The method of claim 82, wherein a 30 nanosecond rise and fall time is present between a peak intensity and a baseline intensity.

93. The method of claim 82, wherein a time between rise and fall times of a peak intensity and a baseline intensity is less than about 10 nanoseconds.

94. The method of claim 82, wherein said electric pulses are applied to said cells in vivo.

95. The method of claim 82, wherein said electrical pulses are applied to said cells in vitro.

96. The method of claim 95 further comprising:
inserting said cells into a subject following applying of said one or more electrical pulses.

97. The method of claim 82, wherein said cells are myoblasts.

98. The method of claim 82, wherein said cells are subject to up to 150 pulses of electricity.

99. The method of claim 98, wherein said cells are subject to five or fewer pulses of electricity.

100. The method of claim 82 further comprising:
administering an additional agent.

101. The method of claim 100, wherein said additional agent is selected from group consisting of an antibiotic compound, an antimicrobial compound, an antibody, a biocidal agent, nanoparticles, self-assembling nanoparticles, viral particles, bacteriophage particles, bacteriophage DNA, genetic material, chemotherapy agent, growth factor, synthetic scaffold, natural scaffold, electrode, drug, a microbe, and a bacteria.
